(19) United States
(12) Patent Application Publication
(10) Pub. No.: US 2008/0208038 A1
(43) Pub. Date: Aug. 28, 2008

(54) Method and Device For Representing the Microstructure of the Lungs

(76) Inventors: Wolfgang Schreiber, Mainz (DE); Ursula Wolf, Darmstadt (DE); Alexander-Wigbert Scholz, Mainz (DE); Claus-Peter Heussel, Mainz (DE)

Correspondence Address: TAYLOR & AUST, P.C., P.O. Box 560, 142. S Main Street, Avilla, IN, 46710, US

(21) Appl. No.: 11/913,445

(22) PCT Filed: Apr. 28, 2006
(86) PCT No.: PCT/EP06/03966

§ 371 Date: Nov. 2, 2007

(30) Foreign Application Priority Data
May 2, 2005 (DE) 10 2005 020 379.5

(51) Int. Cl. A61B 5/055; A61K 49/08

(52) U.S. Cl. 600/420; 424/9.37

(57) ABSTRACT

The invention relates to an apparatus and a method for imaging the microstructure of an animal or human lung by way of introducing a fluoric contrast gas into the lung which is to be imaged; definition of the apparent diffusion coefficient of the contrast gas by way of diffusion weighted ¹⁹fluorine magnetic resonance tomography and based on the determined apparent diffusion coefficients; and imaging of the lung's microstructure.

The current invention also describes a device for the implementation of the inventive method. The science of the current invention allows for the first time the production of a high resolution image of the microstructures of the lung through non-invasive measures, by way of fluorinated gases.

### BACKGROUND OF THE INVENTION

1. Field of the Invention

The invention relates to a method and device for imaging the microstructure of the lung.

2. Description of the Related Art

Different methods are available for the imaging of lung and/or respiratory tract diseases where attempts are made to obtain an original image of the lung and the respiratory tracts. In many instances the available techniques are not precise enough or do not permit sufficient capture of details and their precise resolution. It is therefore an ongoing objective in medical research to find ways to gain better insight into normal or defective developments/conditions of the human body and to aid a physician in his diagnosis by providing data which is as precise as possible.

Various methods are known for the imaging of the microstructure of the lung: pathological changes of the lung are symptomatic for obstructive pulmonary disease COPD (chronic obstructive pulmonary disease) and pulmonary emphysema. COPD is characterized by constriction of airways. Distending lung areas are typical for pulmonary emphysema. Both syndromes are usually diagnosed on the basis of lung function test parameters. The expiratory flow volume reduction (FEV1) is captured by way of a spirometer. However, spirometry testing only provides global information and does not deliver detailed images with detailed resolution. However, information regarding the regional conditions of the airways is crucial for a correct diagnosis and a targeted therapy. According to the latest findings COPD begins in the smallest airways, that is <2 mm (ref. Shaw, R. Djucanovic, D. Tashkin, A. Millar, R. du Bois, P. Corris, “The role of small airways in lung disease”, Respir. Med. 96 (2002) 67-80).

During the course of the disease the greatest significance is attributed to the smallest airways. In addition to the regional procedures, initial clinical tests using magnetic resonance were conducted by way of inhaled hyper-polarized contrast gases. In this method the magnetic resonance tomography (abbreviated “MRT”) of highly polarized noble gases such as ³He-gas has already been established. (ref. K. K. Gast, M. U. Puderbach, I. Rodriguez, B. Eberle, K. Markstaller, A. T. Hanke, J. Schmiedeskamp, N. Weiler, J. Lill, W. G. Schreiber, M. Thelen, H.-U. Kauczor, “Distribution of ventilation in lung transplant recipients: evaluation by dynamic ³He-MRI with lung motion correction”, Invest. Radiol. 37 (2002) 126-134). In addition to ³He-gas initial tests have also been conducted with hyperpolarized ¹²⁹Xe gas for the purposes of medical research.

In both methods the non-polarized gas is initially transferred in a polarized form, usually by way of laser to the tomograph where it is inhaled by the patient. The processing of the gas into a highly polarized contrast gas is very expensive and requires special handling during transportation and application since the artificial polarization condition is destroyed by oxygen and magnetic field exposure. In addition, the costs and the technical expenditure of the methods by way of hyperpolarized noble gases are extremely high.

In addition a series of publications are known where inert fluoric substances, for example fluorine gases such as SF₆, are utilized in order to enable conclusions to be reached with the assistance of ¹⁹fluorine-magnetic resonance (¹⁹F-MRI) regarding function and structure of organs, especially the lung:

According to C. P. Heussel, A. Scholz, M. Schmittner, S. Laukemper-Ostendorf, W. G. Schreiber, S. Ley, M. Quintel, N. Weiler, M. Thelen, H.-U. Kauczor, “Measurements of Alveolar pO₂ Using ¹⁹F-MRI in Partial Liquid Ventilation”, Invest. Radiol. 38, #10, October 2003, pages 635-641 and U. Tokujiro, K. Makita, K. Nakazawa and K. Yokoyama “Relationship between airway pressure and the distribution of gas-liquid interface during partial liquid ventilation in the oleic acid lung injury model: Fluorine-19 magnetic resonance imaging study” Crit Care Med 2000, 28, #8, pages 2904-2908 the “Liquid Ventilation” with perfluorocarbon (PFC) is applied to improve oxygenation. Originally this represents a therapeutic process. At the same time the oxygen partial pressure can be determined from the nuclear spin tomography images. With the exception of the use of fluorinated substances for the purpose of producing lung images, this concept has nothing in common with the current invention.

According to D. O. Kuethe, A. Caprihan, H. M. Gach, I. J. Lowe and E. Fukushima “Imaging obstructed ventilation with NMR using inert fluorinated gases” in J. Appl. Physiol., volume 88, 2000, pages 2279-2286 and D. O. Kuethe, V. V. Behr and S. Begay “Volume of Rat Lungs Measured Throughout the Respiratory Cycle Using ¹⁹F NMR of the Inert Gas SF₆”, Magnetic Resonance in Medicine 48, pages 547-549 (2002) mixtures of oxygen and fluorine gases are inhaled, as is the case in the invention. The cited technology uses a special effect which is seen exclusively in “macroscopic” obstructions. This effect is independent of the diffusion inside the air spaces. It is based on the fact that SF₆ is practically not dissolved in blood (neither are the other fluorine gases) and therefore remains in the alveoli, even though these are well supplied with blood. Oxygen however is dissolved and therefore transported out of the alveoli. If the ventilation is normal then the ratio between ventilation and perfusion is approximately balanced. In the event of obstructed airways, that is reduced ventilation and undisturbed perfusion, more oxygen is removed than replaced. When ventilating with SF₆ and oxygen of medium SF₆ concentration (for example 40%) a concentration effect of SF₆ and thereby an increase in the signal intensity will result after a while. This concentration effect is the basis of the image creation as described by Kuethe et al. and does not describe the science of the current invention.

The publication “Dynamic ¹⁹F-MRI of Pulmonary Ventilation Using Sulfur Hexafluoride (SF₆) Gas” by W. G. Schreiber, B. Eberle, S. Laukemper-Ostendorf, K. Markstaller, N. Weiler, A. Scholz, K. Bürger, C. P. Heussel, M. Thelen and H.-U. Kauczor in Magnetic Resonance in Medicine 45, pages 605-613 (2001) describes dynamic fast image creation of the lung by way of SF₆ which can be utilized for functional, and to a limited extent also for anatomical questions. Diffusion-dependent effects are also not used here. The sequential components are so short that no diffusion effect with the fluorine gases, which diffuse very slowly, is observed.

In addition U.S. Pat. No. 6,574,497 B1 refers to the use of ¹⁹fluoric substances as contrast agents and markers for medical devices and is conceived for an entirely different field of application than the present invention—that is for the control of various cardiovascular interventions such as angiography. It addresses devices which explicitly do not contain fluorine gas and which are intended to be placed in blood vessels.

European Patent No. EP 0 599 946 B1 discloses a method for rendering ¹⁹fluorine-magnetic resonance images of organs and tissue whereby an organ and tissue of a mammal is administered with a diagnostically effective amount of a perfluorinated carbon cluster in a pharmaceutically acceptable medium and whereby the perfluorinated carbon cluster has the formula CₙFₘ whereby n is in the range of approximately 30 to approximately 100 and m is ≦n and (b) the organs and tissues are imaged. The described fluorinated carbon clusters with a high molecular weight cannot be considered for lung imaging. It is certain that the diffusion of clusters of this type cannot be proven in any form whatsoever in any feasible imaging situation.

What is needed in the art are methods and devices which have improved usability in the medical field.

### SUMMARY OF THE INVENTION

The present invention provides a non-invasive method or device, with which the images of the lung can be captured and reproduced, also in regional areas, in detailed resolution. Accordingly, the current invention provides means with which even the smallest constrictions or distensions of the lung and/or the airways can be imaged at an early stage and can thereby be detected, thereby providing an opportunity to prevent, or to therapeutically intervene, as early as possible, lung diseases like COPD (chronic obstructive pulmonary disease) and lung emphysema. In addition, the high costs and expenditure associated with the methods and devices that are known from the current state of the art are avoided.

The present invention, in one form provides a method for imaging of an animal or a human lung's microstructure including the steps of:
introducing a fluoric contrast gas into the lung which is to be imaged;
definition of the apparent diffusion coefficient apparent diffusion coefficient (ADC) of the contrast gas by way of diffusion weighted ¹⁹fluorine magnetic resonance tomography and based on the determined apparent diffusion coefficients; and
imaging of the lung's microstructure.

The present invention in another embodiment includes a device for imaging of an animal's or a human's lung's microstructure having a ¹⁹fluorine magnetic resonance tomograph which is equipped with apparati to determine the diffusion of contrast gas in order to image the gas-filled spaces of the lung.

The current invention is described in detail below, whereby the explanatory comments regarding the device correspondingly also apply equally to the method, and vice versa.

Due to the low tissue density of the lung as well as due to intro-pulmonary magnetic field non-homogeneities magnetic resonance tomography by way of protons is not well suited for lung imaging. In contrast, the current invention successfully allows imaging of the gas-filled spaces of the lung by utilizing contrast gases and the diffusion effects which are associated with these. In other words, based on data, determined by a computer, a relevant image or partial image of selected areas of the lung is generated. In accordance with the present invention a diffusion-weighted contrast gas—¹⁹fluorine magnetic resonance tomography is used which utilizes the effect that the incoherent movement of the gas molecules (diffusion) causes a signal deletion through locally dependent magnetic fields (gradient fields). With firm test parameters the level of the deletion depends essentially on the diffusion coefficient of the gas. With constricted gas spaces, for example, respiratory tracts, which are obstructed due to COPD the contrast gas molecules or atoms push against the adjacent structures, for example the bronchial walls. A reduced, so-called “apparent diffusion coefficient” (ADC) is measured. With firm gradient parameters this is first and foremost dependent upon the diameter of the gas spaces and is therefore especially suitable for the capture and imaging of small, as well as large spaces, such as constricted or distending lung areas.

In the current invention the term “diffusion-weighted ¹⁹fluorine-magnetic resonance tomography” is to be understood to be the already known ¹⁹fluorine-magnetic resonance tomography, which is modified so that diffusion measurements could be conducted on the fluorinated gases which serve as the basis for calculations to be able to image the microstructure of a lung.

In accordance with the current invention the ADC are therefore determined by way of magnetic resonance tomography, whereby the smallest airway constrictions and distensions can be captured and imaged regionally, non-invasively in detailed resolution. Within the scope of the current invention “non-invasive” means that neither permanent changes of the body or the organs, especially the lung are caused by the method or the device, nor that any harmful side effects occur. The inventive method and device have no effect on the body or the organ which is to be imaged.

With highly polarized ³He-gas or ¹²⁹Xe-gas the measuring principle is essentially analogous to the diffusion measurement. Nevertheless, no gas processing whatsoever is necessary. In addition, the especially fast relaxation with fluorinated gases further assumes provisions for measuring the diffusion before the signal is irretrievably lost. This means that the echo time (TE) is selected in the order of magnitude of the transversal relaxation time T₂. A substantial advantage of the equally short longitudinal relaxation time T₁ of the gases that are to be measured, such as fluorine gasses, exists in that a high number of signal averagings can be made. This results in a favorably signal/noise ratio for measuring times which are acceptable for a living organism.

According to an embodiment of the present invention the determination of the apparent diffusion coefficient (ADC) occurs by way of ¹⁹fluorine magnetic resonance tomography, which has been proven to be especially suitable for imaging of the microstructure of the lung and consequently as a basis for detection of changes in the microstructure of the lung. In accordance with the current invention, harmless non-toxic inert gases, especially fluorine gases, are used which have no therapeutic or other effect whatsoever, which are preferably inert and which are known to be totally non-toxic in appropriate application. In accordance with the current invention “fluorine” gases are to be understood to be perfluorinated gases which, due to their inert character and their high level of stability, do not enter into or cause any reactions and which are therefore totally harmless for the human or animal body. Perfluoralkanes are cited as an example. In addition to other applications these are also used as blood substitutes or as gases in ophthalmology, thereby confirming their safeness for an organism.

Fluorine gases possess relatively high molecular weights (approximately 80-200 g/mol) and therefore low diffusivity. Since the “dimensional resolution” depends directly on the diffusivity and diffusion time, the smallest structures can be explored with the slow fluorine gases, whereas these cannot be captured by helium gas which diffuses approximately 6 to 8 times faster. The current invention therefore concerns a method and a device for imaging the microstructure of the lung, which are sensitive to the dimensional changes of the air spaces.

The recording technique includes sequential components which produce explicit diffusion-weighted effects, which are authenticated when compared with a reference, specifically a reference image, where these components are turned off. Sequential components are periodically repeated gradient fields which are arranged such that their effect upon stationary atoms or molecules terminates, that diffusing atoms or molecules however experience a net de-phasing of the spins and a signal drop. Bipolar gradient connections can, for example, be used for this.

The inventive method of the diffusion-weighted ¹⁹fluorine magnetic resonance tomography is accomplished in a conventional clinical tomograph which, for example, has a basic field of 1.5 T. However, other field intensities can be used, for example 0.2 T through 3 T. For the inventive method or the inventive device a conventional tomography could, for example, be converted so that it can transmit and receive on the Larmor frequency of ¹⁹F. In addition, a coil is required which transmits and receives on the frequency of the fluorine nucleus (Larmor frequency). Appropriately designed software can, for example, be used as a way to determine the diffusion of the utilized gases.

For example, the present invention starts with a conventional magnetic resonance tomograph which customarily permits proton imaging and which can also be used for highly polarized noble gases. The tomograph is converted in order to determine the various diffusion effects of inert fluorine gases by way of the diffusion coefficient at given diffusion gradients in a lung. A magnetic resonance tomograph, which is equipped to measure highly polarized noble gases, such as ³He-gas and which is converted according to the invention, as previously described, can also be used as a starting device. Measuring of the diffusion gradients and determination of diffusion coefficients so that an appropriate conversion of a magnetic resonance tomograph is easily possible.

A comparison of a reference image, with an image of the lung, possibly in the form of a three-dimensional recreation, can be produced, with appropriate software on the monitor. This is done with the aid of the obtained data set and reevaluation of the data, whereby various image options are available, depending upon the software.

Various inert fluorine gases may be used as contrast gas. Especially suitable are perfluoroalkanes and perfluorosulphur hydrides; for example especially CF₄, C₂F₆, C₄F₈, C₃HF₇ (heptafluoropropane) and SF₆. No toxicity is known for any of these gases when used appropriately. They can be administered without hesitation as being non-toxic. The contrast gas is usually breathed in or administered as a fluorine gas/breathable mixture or as a fluorine gas/oxygen mixture. A fluorine gas/breathable air mixture, which is enriched with the desired oxygen content can also be used as a contrast gas. Oxygen is included in order to provide a concentration that is approximately equal to breathable air. It is especially advantageous to add the physiological oxygen concentration, which is normally breathed in by the body to the contrast gas, that is in the range of 20 to 80 weight-%, especially approximately 20 weight-% oxygen content, so that any asphyxiating effects are eliminated generally from the outset.

In the present scenario “contrast gas” refers to the measuring gas, which has no association with a contrast agent as such. Inhaling of totally non-toxic gases, such as a fluorine/oxygen mixture, is comparable to inhaling of a gas mixture, which would be used for diving and which is also non-invasive and does not have any effect on the organism.

The contrast gases, which are used according to the current invention, have an advantage in that the costs and technological expenditure of the methods which are known from the current state of the art that use hyperpolarized gases are clearly reduced, since much cheaper fluorine gases are utilized, which do not need to be polarized. It is however also possible to utilize fluorine gas in a polarized form, not necessarily in hyperpolarized form, whereby the expenditure is clearly less when compared to the aforementioned highly polarized noble gases.

The gas mixture including fluorine gas and breathable air, fluorine gas and oxygen or fluorine gas, breathable air and oxygen can be administered by way of an applicator unit, which is hand operated or computer controlled. If a conventional breathing respirator is used, then the physiological characteristics of the fluorine gases must be considered in order to be able to apply them as volume-controlled. It is preferable if a volume-controlled quantity of contrast gas, especially a fluorine gas/breathable air and/or fluorine gas/oxygen mixture, is supplied or inhaled. The supplied volume of gas can however also be supplied in any other controlled manner or method for example pressure controlled. In accordance with a preferred embodiment of the present invention it is functional if the contrast gas is utilized at a regulated constant breathing frequency. Volumes in the range of 200 to 600 ml/breath, especially 300 ml/breath are especially preferred.

The inventive device or the inventive method are advantageously operated or implemented so that the measurement of the apparent diffusion coefficient (ADC) occurs in synchronization with the breathing, preferably continuously; this may, for example, be accomplished by triggering or holding ones breath.

It has been proven to be advantageous if the fluorine gas with oxygen is used in the range of 2:8 to 8:2, especially 8:2. It is especially preferred if the measuring gas, such as a fluorine gas/breathable air and/or oxygen mixture, is supplied at a predetermined firm ratio.

In accordance with an especially preferred embodiment of the present invention the fluorine gas/breathable air and/or oxygen mixture is supplied at a volume-controlled rate of 300 ml/breath at a mixture ratio of 8:2 at a constant breathing frequency.

In addition to the aforementioned gases, other gases can also be additionally used due to the measuring techniques utilized by the present invention. These may, for example, be enlisted to effect a change in the diffusion coefficient of the entire mixture. These additional gases must of course also be able to be administered totally safely and be non-toxic to an animal or human.

For the purpose of lung imaging, especially in animals such as domestic pigs, it could be especially useful to steady the lung, for example to conduct the procedure under anesthesia. In such an instance the vital functions are usually monitored. The measurements can however also be conducted on waking animals or humans; in this scenario monitoring of the oxygen saturation, for example by way of a non-invasive pulse oximeter is recommended. In a pulse oximeter the oxygen saturation is measured and evaluated with the assistance of a photo electrode through the spectral absorption of the hemoglobin. This occurs blood-free and totally painless on body parts which are well supplied with blood, such as fingers or ear lobes. Measuring and data collection according to the inventive method and the operation of the inventive device can be implemented by a person without medical training and without medical knowledge and expertise.

According to the current invention it is especially preferred if a gradient echo sequence is utilized for a determination of the diffusion weighted magnetic resonance sequences. However, other sequence types such as Spinecho, or Steady-State-Free-Prescession can also be utilized. In addition to the image producing gradients, a bipolar diffusion gradient is implemented in at least one direction in space, for example, in direction of the layer selection.

The apparent diffusion coefficient (ADC) is essentially dependent upon the magnetic resonance signal intensities and the so-called b-value which is calculated from the parameters of the gradient. The gradient factor b describes the gradient strength, the time for turning on and off, and the time history of the gradient fields.

The determination of the apparent diffusion coefficient (ADC) along one direction in space (one-dimensional) occurs through the quotient from the signal intensity under influence of the diffusion gradient SI(b) and the signal intensities with turned off diffusion gradient (amplitude=0) SI(0) according to the following formula:

SI(b)/SI(0)=exp(−b×ADC)

The signal intensity is preferably determined according to the image spots in the diffusion weighted image and the reference image. In accordance with the current invention, a b-value dependent diffusion effect can be authenticated in vivo.

It is of course also possible to determine the apparent diffusion coefficient (ADC) as a tensor through the combination of diffusion gradients in several directions in space.

The diffusion coefficients are determined through the diffusion gradients in a manner so that the additional measuring parameters, which need to be adjusted, are appropriately predefined and therefore established in a suitable manner in order to be able to measure the diffusion. Especially the gradient factor b is preset. It is of course also possible to adjust a plurality of gradient factors.

The evaluation of the obtained data is done in comparison with a reference, for example, a reference image where the diffusion gradient is turned off, whereby commercially available software may be used to implement the comparison. The comparison is made with and without diffusion-dependent signal reduction, with identical recording parameters at the same anatomical location. This allows the production of a quasi-image of the lung, enabling a physician to determine various symptoms such as obstructions and distension based on the images.

In another embodiment of the present invention the apparent diffusion coefficient is measured as a quasi-absolute value and compared with a reference without diffusion. Direct conclusions regarding the microstructure of the lung can be reached and it is also possible to rely upon the apparent diffusion coefficient by comparison of the values from several measurements as a basis to determine changes in the microstructure of the lung. Therefore, several measurements can be conducted of the apparent diffusion coefficient in dependency upon the utilized volume of contrast gas. The evaluation of the data then occurs, for example, through comparison of two measurements with identical recording parameters and at the same anatomical location on the same individual, however using varying volumes of contrast gas.

It is especially preferred if a smaller volume of contrast gas is used in a first measurement, and a larger volume in a second measurement, each time measuring the apparent diffusion coefficient. The respectively utilized volumes preferably differ clearly from each other. As a rule of thumb, the larger volume would differ from the smaller volume by as much as 2 to 4 times. The first smaller volume may, for example, have a defined value in the range of 100 to 250 ml and the second larger volume a defined value in the range of 200 to 1,000 ml. It is especially preferred, if the residual volume after breathing out, or the functional residual capacity (FRC) is selected as the first, smaller volume. Each measurement is appropriately taken several times, each time determining the mean value of the measured apparent diffusion coefficient.

In one embodiment of the present invention the determination of the apparent diffusion coefficient occurs in dependency on the volume. In order to achieve defined lung volumes, breathing out is initiated after having introduced or breathed in the contrast gas, causing the residual gas volume or the so-called functional residual capacity FRC to remain in the lung. The apparent diffusion coefficient (ADC-values) of the contrast gas is measured with this residual gas volume (first volume). Several measurements are preferably conducted and the mean value is determined from the obtained measurements.

Subsequently, a second control volume with a defined amount of gas of fluoric contrast gas is brought into or breathed into the lung that is to be imaged second volume and the apparent diffusion coefficient (ADC-value) of the contrast gas is again measured. Also measurements are taken preferably several times, establishing the mean value from these. The two volumes may be selected such that they distinguish themselves clearly from each other, so that each time the measurements of the apparent diffusion coefficient are taken for the first and second supplied volume.

The microstructure of the airways in the lung is captured by comparing the apparent diffusion coefficients of both conditions with varying volumes. Depending on the volume of the utilized contrast gas, a significant change of the apparent diffusion coefficient results, allowing a conclusion regarding the size of the air-filled spaces in the microstructure of the lung. This means that based on the change, one can calculate back to the microstructure, definitively rendering an image of the change in the microstructure of the lung.

The diffusion weighted magnetic resonance tomography accordingly leads to information regarding the microstructure of the lung, since changes of the apparent diffusion coefficient ADC are associated with the microstructure of the lung. For example, pathologically expanded airways which are characteristic for pulmonary emphysema, display a clearly increased apparent diffusion coefficient.

If the administration of varying volumes of contrast gas leads to a significant change, for example an increase of the measured ADC values of the administered gas, an image of the changes in the microstructure of the lung can be established. This allows for the change in the microstructure of the lung to be imaged, at least in partial sections.

It is however preferable to conduct the inventive process with only one controlled volume.

The method and the device of the present invention is designed for imaging of the lung for the living animal, for example a domestic pig or the living human being, as well as for use with dead organisms. Pathological determinations are also possible, whereby the diffusion effects for various distension conditions of pathological size changes in the healthy or the diseased lung, for example, obstructive pulmonary diseases such as COPD and pulmonary emphysema, can be established in the same manner.

The method of the present invention, as well as the device of the current invention are intended for data collection only and provides provisional results which can only be attributed to a certain syndrome in a subsequent step by a physician. It does not represent a diagnostic procedure. The inventive method for determining the physical apparent diffusion coefficient in the living and/or dead human or animal body alone does not provide a determination regarding a necessary medical treatment.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1a is an in-vivo image of a coronary sectional plane of a pig's lung, which is filled with C₄F₈ gas, with diffusion-dependent signal reduction, in accordance with an embodiment of the present invention, whereby the diffusion gradient is vertical to the focal plane; and

FIG. 1b is an in-vivo image of a coronary sectional plane of a pig's lung, which is filled with C₄F₈ gas, without diffusion dependent signal reduction, which is not in accordance with the current invention, with identical image parameters and at the same anatomical position.

Corresponding reference characters indicate corresponding parts throughout the several views. The exemplification set out herein illustrates one embodiment of the invention, in one form, and such exemplification is not to be construed as limiting the scope of the invention in any manner.

### DETAILED DESCRIPTION OF THE INVENTION

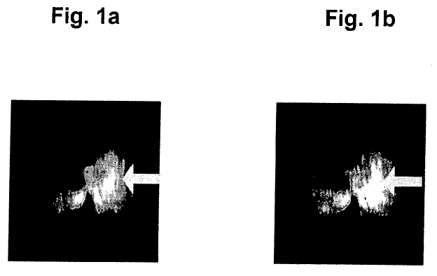

Referring now to the drawings, in the diffusion-weighted image in FIG. 1a, a signal reduction can be noted when compared with a non-diffusion-weighted image with a magnetic resonance tomograph as illustrated in FIG. 1b. This is a result of the diffusion of the C₄F₈, which is being brought into the airways of the lung in this example. This enables the apparent diffusion coefficients to be determined with which a realistic partial image of a lung, including the microstructures can be produced in high resolution.

The advantages of the inventive science are multifaceted: A method and a device are provided in accordance with the current invention which, compared to the current state avoid the high cost of noble gases, and the technological expenditure necessary to polarize these gases.

With the inventive non-invasive method and non-invasive device the smallest airway constrictions and distensions are regionally captured and images provided at a high resolution by way of utilizing totally nontoxic contrast gases and by way of determining the apparent diffusion coefficient (ADC).

Also advantageous are the short relaxation times of the measuring gases, especially the fluorine gases, which allows for a high number of signal messages and results in a favorable signal/noise ratio, especially for a measuring time that is acceptable for a living organism.

The measured values of the apparent diffusion coefficient are determined directly, preferably in comparison with a reference without limitation of the diffusion. Or a change of the apparent diffusion coefficient, in dependency upon the contrast gas volume through comparison of two measurements, serves as a basis for clarification and image presentation of the changes in the microstructure of the lung.

The present invention also enables microstructures of the lung, including regional areas to be captured and reproduced in high resolution in order to enable a physician to reach better conclusions regarding pathological conditions, especially obstructive respiratory tract diseases and pulmonary emphysema. Even the smallest constrictions or distensions of the lung and/or airways can be imaged at an early stage and can thereby be discovered in order to prevent or to therapeutically intervene as early as possible in pulmonary diseases such as COPD (chronic obstructive pulmonary disease) and pulmonary emphysema.

One can revert to technology already existing in the current state of the art, especially the ¹⁹fluorine magnetic resonance tomography, for the technical sequence of producing and capturing of resonance signals. However, the present invention provides the physician with more precise data allowing him to derive a sound diagnosis and efficient treatment.

For example, the following trial of the method is discussed and an illustration of the use of the present invention. This trial is not the sole use of the invention, but it is illustrative only.

### Trial:

Objective:
The objective of the present study is the determination of the apparent diffusion coefficient ADC of C₄F₈ in a pig's lung at a controlled lung volume

Material and Methods:
Coronary diffusion-weighted images were recorded on 4 anesthetized pigs (median weight 20 kg) by way of 2D-FLASH-Sequence (TE/TR/NEX/α=8.7; 8.1 ms/16; 15.6 ms/28; 30/40°) at 1.5 T by way of Birdcage-coil, a raw data matrix of 64×128 and at a bandwidth of 104 Hz/pixel after infusion of 20% O₂-80%-C₄F₈ gas mixtures. Repeated measurements with a measuring time of 59 s at a b-value of 49.84 s/cm² per animal were taken, consistent with the science of holding ones breath, after expiration.

Results:
Diffusion-dependent signal deletion in the animal lung could repeatedly be proven for example see FIGS. 1a and b. A median ADC of 0.128+0.003 cm²/s resulted in the residual volume FRC (functional residual capacity) after exhalation.

Conclusion:
The intrapulmonary ADC of C₄F₈ was determined for the first time in-vivo while holding the breath in an animal. The utilization of the ¹⁹F-MRT for the examination of the microstructure of the lung is recommended as a technologically uncomplicated alternative to the already known methods in which highly polarized gases such as ³Helium are used which are expensive to process.

FIGS. 1a and 1b illustrate the coronary images of a pig's lung which were obtained in the tests after ventilation with an 80% C₄F₈-20% O₂ mixture while holding the breath with FRC:
FIG. 1a with turned on diffusion gradient
FIG. 1b without turned on diffusion gradient

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1-44. (canceled)

45. A method for imaging the microstructure of one of an animal and a human lung, comprising the steps of:
introducing a fluoric contrast gas into the lung that is to be imaged;
definition of an apparent diffusion coefficient of said fluoric contrast gas by way of diffusion-weighted ¹⁹fluorine magnetic resonance tomography and based on a determination of apparent diffusion coefficients; and
imaging of microstructure of the lung.

46. The method of claim 45, wherein said contrast gas is breathed into the lung.

47. The method of claim 45, wherein said fluoric contrast gas is selected from a group of perfluoroalkanes and perfluorosulphur hydrides.

48. The method of claim 47, wherein said fluoric contrast gas is one of CF₄, C₂F₆, C₄F₈, C₃HF₇ and SF₆.

49. The method of claim 45, wherein said fluoric contrast gas is at least one of a fluorine gas/breathable air mixture and a fluorine gas/oxygen mixture.

50. The method of claim 45, wherein said fluoric contrast gas includes a physiological oxygen concentration.

51. The method of claim 50, wherein said physiological oxygen concentration includes oxygen in said contrast gas in a range of 20 to 80 weight-%.

52. The method of claim 51, wherein in said oxygen is approximately 20 weight-% of said contrast gas.

53. The method of claim 49, wherein said fluorine gas is utilized with oxygen at a ratio in the range of 2:8 to 8:2.

54. The method of claim 53, wherein said ratio is approximately 8:2.

55. The method of claim 45, wherein said contrast gas is supplied in a predetermined ratio.

56. The method of claim 45, wherein said contrast gas is supplied at a volume controlled rate at an adjusted substantially constant breathing frequency.

57. The method of claim 56, wherein said contrast gas is at least one of a fluorine gas/breathable air and a fluorine gas/oxygen mixture that is supplied in a range of 200 ml/breath to 600 ml/breath.

58. The method of claim 57, wherein said mixture is supplies at approximately 300 ml/breath.

59. The method of claim 48, wherein at least one of said fluorine gas/breathable air and said fluorine gas/oxygen mixture is supplied at a volume controlled rate of approximately 300 ml/breath in a mixture ratio of approximately 8:2 at a substantially constant breathing frequency.

60. The method of claim 45, wherein said determination of said apparent diffusion coefficient occurs in substantial synchronization with breathing.

61. The method of claim 45, wherein a gradient echo sequence is utilized for the ¹⁹fluorine magnetic resonance tomography.

62. The method of claim 45, wherein said apparent diffusion coefficients are determined with predefined established measuring parameters.

63. The method of claim 62, wherein said predefined established measuring parameters is a preset gradient factor b.

64. The method of claim 45, wherein said apparent diffusion coefficient is determined by way of a bipolar diffusion gradient in at least one direction in space.

65. The method of claim 64, wherein said apparent diffusion coefficient is determined as a tensor by way of a bipolar diffusion gradient in several directions in space.

66. The method of claim 45, further comprising the step of evaluating data that occurs through a comparison of a reference with identical recording parameters and at the same anatomical location where no diffusion occurs.

67. The method of claim 45, wherein said apparent diffusion factor is determined depending upon a utilized volume of said contrast gas.

68. The method of claim 67, further comprising the step of taking several measurements of said apparent diffusion factor with different volumes of said contrast gas.

69. The method of claim 68, further comprising the step of evaluating data that occurs by comparing two measurements with identical recording parameters and at the same anatomical location using varying volumes of said contrast gas.

70. The method of claim 69, wherein a first measurement of said two measurements a smaller volume and in a second measurement of said two measurements a larger volume of said contrast gas is used.

71. The method of claim 70, wherein one of a residual volume after breathing out and a functional residual capacity (FRC) is selected as said smaller volume.

72. The method of claim 70, wherein said larger volume is from 2 to 4 times the size of said smaller volume.

73. The method of claim 67, further comprising the steps of:
taking a plurality of measurements; and
determining the mean value of said apparent diffusion coefficient from said plurality of measurements.

74. The method of claim 45, wherein said apparent diffusion coefficient (ADC) is determined along one direction in space according to the following formula:
SI(b)/SI(0)=exp(−b×ADC)
where:
SI(b) is a signal intensity with turned on diffusion gradient;
SI(0) is a signal intensity with turned off diffusion gradient (amplitude=0); and
b is a b-value which is calculated from the parameters of the gradients.

75. A device for imaging the microstructure of one of an animal and a human lung, comprising a ¹⁹fluorine magnetic resonance tomograph which is equipped with an apparatus to determine a diffusion of a contrast gas in order to produce an image of gas-filled spaces in the lung.

76. The device in accordance with claim 75, further comprising an applicator unit for said contrast gas which is calibrated for said contrast gas.

77. The device of claim 76, wherein said applicator unit for said contrast gas operates at a volume-controlled rate at an adjusted substantially constant breathing frequency.

78. The device of claim 77, wherein said applicator unit is adjusted to a throughput rate (tidal volume) in the range of 200 to 600 ml/breath.

79. The device of claim 78, wherein said tidal volume is approximately 300 ml/breath.

80. The device of claim 75, wherein said contrast gas includes a fluorine gas which is selected from the group consisting of perfluoroalkanes and perfluorosulphur hydrides.

81. The device of claim 80, wherein said fluorine gas is one of CF₄, C₂F₆, C₄F₈, C₃HF₇ and SF₆.

82. The device of claim 76, wherein said contrast gas includes at least one of a fluorine gas/breathable air mixture and a fluorine gas/oxygen mixture.

83. The device of claim 82, wherein said contrast gas is pre-mixed to a pre-established ratio.

84. The device of claim 83, wherein said applicator unit provides a fluorine gas/oxygen with said pre-established ratio being in the range of 2:8 through 8:2.

85. The device of claim 84, wherein said ratio is 8:2.

86. The device of claim 76, wherein said contrast gas includes a physiological oxygen admixture in the range of 20 to 80 weight-% of said contrast gas.

87. The device of claim 76, wherein said applicator unit provides at least one of a fluorine gas/breathable air and oxygen mixture at a volume controlled rate of approximately 300 ml/breath at a mixture ratio of 8:2 at a substantially constant breathing frequency.

88. The device of claim 87, wherein said apparatus for determination of said apparent diffusion coefficient operates in synchronization with breathing.

89. The device of claim 87, wherein said ¹⁹fluorine-magnetic resonance tomograph functions on the basis of a gradient echo sequence.

90. The device of claim 89, wherein the device is configured to use measuring parameters that are preset constantly for determination of said apparent diffusion coefficients.

91. The device of claim 90, wherein said measuring parameters include a gradient factor b.

92. The device of claim 91, further comprising an apparatus by which said apparent diffusion coefficient is determined by way of a bipolar diffusion gradient in at least one direction in space.

93. The device of claim 92, wherein said apparatus determines said apparent diffusion coefficient as a tensor by way of said bipolar diffusion gradient in several directions in space.

94. The device of claims 76, further comprising an evaluation unit that conducts a comparison of obtained data with a reference having identical recording parameters at the same anatomical location except that no diffusion occurs.

95. The device of claim 76, further comprising an evaluation unit for obtained data, said evaluation unit conducts a comparison of values of said apparent diffusion coefficient between two measurements in dependency on a volume of said contrast gas with identical recording parameters and at the same anatomical location.

96. The device of claims 76, further comprising an apparatus by which said apparent diffusion coefficient (ADC) is determined along one direction in space according to the following formula:
SI(b)/SI(0)=exp(−b×ADC)
where:
SI(b) is a signal intensity with turned on diffusion gradient;
SI(0) is a signal intensity with turned off diffusion gradient (amplitude=0); and
b is a b-value which is calculated from the parameters of the gradients.

97. A medical apparatus, comprising a diffusion-weighted contrast gas 19fluorine-magnetic resonance tomograph for imaging the microstructure of one of an animal and a human lung.
